(19) United States
(12) Patent Application Publication
(10) Pub. No.: US 2007/0225680 A1
(43) Pub. Date: Sep. 27, 2007

(54) GUIDING CATHETER WITH CHEMICALLY SOFTENED DISTAL PORTION AND METHOD OF MAKING SAME

(75) Inventor: James F. Biggins, Waltham, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/277,051

(22) Filed: Mar. 21, 2006

(51) Int. Cl. A61M 25/00; B32B 37/00

(52) U.S. Cl. 604/526; 604/530; 264/514

(57) ABSTRACT

A guiding catheter for placement in a patient's vessel. The catheter includes an elongate hollow shaft with open proximal and distal ends and a bore extending there through. The catheter shaft includes an inner liner, a metallic reinforcement layer overlying the inner liner, and a unitary outer jacket covering the reinforcement layer. A distal portion of the outer jacket of the catheter shaft is chemically softened to be more flexible than a proximal portion of the outer jacket. A connector fitting is mounted at the proximal end of the shaft in communication with the bore and a distal tip is attached to the distal end of the shaft. A method of manufacturing the guiding catheter is also disclosed.

### FIELD OF THE INVENTION

The present invention relates generally to an intraluminal guiding catheter used in a medical procedure, and more particularly, to a guiding catheter with a chemically softened distal portion and a method of making same.

### BACKGROUND OF THE INVENTION

A stenosis, or narrowing of a blood vessel such as a coronary artery may comprise a hard, calcified substance and/or a softer thrombus material. There have been numerous therapeutic procedures developed for the treatment of stenosis in a coronary artery. One of the better-known procedures is percutaneous transluminal coronary angioplasty (PTCA). According to this procedure, the narrowing in the artery can be reduced by positioning a dilatation balloon across the stenosis and inflating the balloon to re-establish acceptable blood flow through the artery. Additional therapeutic procedures may include stent deployment, atherectomy, and thrombectomy, which are well known and have proven effective in the treatment of such stenotic lesions.

The therapeutic procedure starts with the introduction of a guiding catheter into the cardiovascular system from a convenient vascular access location, such as through the femoral artery in the groin area or other locations in the arm or neck. The guiding catheter is advanced through the arteries until its distal end is located near the stenosis that is targeted for treatment. During PTCA, for example, the distal end of the guiding catheter is typically inserted only into the ostium, or origin of the coronary artery. A guidewire is advanced through a central bore in the guiding catheter and positioned across the stenosis. An interventional therapy device, such as balloon dilatation catheter, is then slid over the guidewire until the dilatation balloon is properly positioned across the stenosis. The balloon is inflated to dilate the artery. To help prevent the artery from re-closing, a physician can implant a stent inside the artery. The stent is usually delivered to the artery in a compressed shape on a stent delivery catheter and expanded by a balloon to a larger diameter for implantation against the arterial wall.

In order for the physician to place the guiding catheter at the correct location in the vessel, the physician must apply longitudinal and rotational forces. In order for the guiding catheter to transmit these forces from the proximal end to the distal end, the catheter must be rigid enough to push through the blood vessel, a property sometimes called pushability, but yet flexible enough to navigate the bends in the blood vessel. The guiding catheter must also have sufficient torsional stiffness to transmit the applied torque, a property sometimes called torqueability. To accomplish this balance between longitudinal rigidity, torsional stiffness, and flexibility, there is often a support member added to the catheter shaft. This support member is often comprised of a reinforcing braid or coil embedded in the shaft. This support wire is often embedded in the shaft between the two layers of tubing that comprise the shaft.

Using the femoral artery approach in a PTCA procedure, a guiding catheter is passed upward through the aorta, over the aortic arch, and down to the ostium of the coronary artery to be treated. It is preferable the catheter have a soft tip or flexible section for engaging the ostium of the selected branch vessel. Therefore, it is advantageous to have the proximal section be rigid to transmit the forces applied, but to have the distal end more flexible to allow for better placement of the guiding catheter. The need for this combination of performance features makes it desirable for a guiding catheter shaft to have variable flexibility along the length of the catheter. More specifically, it is desirable for a guiding catheter to have increased flexibility near the distal end of the catheter shaft and greater stiffness near the proximal end.

One approach used to balance the need for pushability and torqueability while maintaining adequate flexibility has been to manufacture a guiding catheter that has two or more discrete tubular portions over its length, each having different performance characteristics. For example, a relatively flexible distal section may be connected to a relatively rigid proximal section. When a guiding catheter is formed from two or more discrete tubular members, it is often necessary to form a bond between the distal end of one tubular member and the proximal end of another tubular member. This method requires substantial manufacturing steps to assemble the various sections and makes it difficult to manufacture the entire shaft utilizing coextrusion technology. Further, the shaft design may include relatively abrupt changes in flexibility at material changes.

Various approaches for achieving variable stiffness of the guiding catheter shaft include varying the braid pitch of the reinforcing layer and/or by varying the properties of materials used in construction, such as by removing a selected distal portion of an outer tubular layer of the catheter shaft and replacing that distal portion with one or more sections of more flexible tubing. A unitary catheter shaft arrangement with variable stiffness is also known that incorporates one or more layers of a material that is selectively curable by ultraviolet light, wherein selected portions of the catheter shaft are subjected to radiation to cure the material and thereby increase the stiffness of the shaft in the treated area.

However a need still exists for guiding catheter shafts that can be easily manufactured, such as by extrusion, and yet are capable of having a variable stiffness without assembling multiple components of the shaft or attending to difficulties inherent in irradiated variable-stiffness catheters, such as the limitations in the choice of catheter materials and in the control of the final catheter properties.

### BRIEF SUMMARY OF THE INVENTION

An embodiment of the present invention is a guiding catheter for placement in a patient's vessels, such as the vasculature. The catheter includes an elongate hollow shaft with open proximal and distal ends and a bore extending there through. The shaft includes an inner liner, a reinforcement layer overlying the inner liner, and a unitary outer jacket covering the reinforcement layer. In various embodiments, a distal portion of the outer jacket and/or inner liner of the shaft are chemically softened to be more flexible than a proximal portion of the outer jacket and/or inner liner. In various embodiments of the present invention, a connector fitting is mounted at the proximal end of the shaft in communication with the bore, and/or a soft distal tip is attached to the distal end of the shaft.

In another embodiment, the chemically softened distal portion of the outer jacket of the catheter shaft includes a first softened segment with a first flexibility and a second softened segment with a second flexibility greater than the first flexibility to provide the catheter shaft distal portion with an increase in flexibility as it extends distally.

Another embodiment of the present invention is a method of manufacturing a guiding catheter with variable flexibility along a length of the catheter shaft. The method includes forming a catheter shaft subassembly by extruding a first material to form a tubular liner, braiding a reinforcement layer over the tubular liner, and extruding a second material over the reinforcement layer to form an outer jacket. A distal portion of the catheter shaft subassembly is then submerged into a softening agent to chemically soften the outer jacket and/or the inner liner to thereby increase the flexibility of the distal portion. After a suitable amount of time, the distal portion of the catheter shaft is removed from the softening agent, and, optionally, wiped or cleaned of any remaining softening agent. In various embodiments of the present invention, a connector fitting is mounted at the proximal end of the shaft in communication with the bore, and/or a soft distal tip is attached to the distal end of the shaft to construct the guiding catheter.

In another embodiment, the step of removing the distal portion from the softening agent includes removal of a first segment of the distal portion after a first time period, such that a second segment of the distal portion remains submerged until expiration of a second time period.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a guiding catheter according to an embodiment of the present invention positioned within a patient's vascular system.

FIG. 2 illustrates a side view of the guiding catheter of FIG. 1.

FIG. 3 is a transverse cross-sectional view of the guiding catheter of FIG. 2 taken along line 3-3.

FIG. 4 is a longitudinal sectional view of a distal portion of the guiding catheter of FIG. 2.

FIG. 5 schematically illustrates a method of manufacturing a guiding catheter in accordance with an embodiment of the present invention.

FIG. 6 is a graph of load as a function of degrees of deflection for catheter shafts softened in accordance with various embodiments of the present invention.

FIG. 7 illustrates a distal portion of a catheter shaft softened in accordance with an embodiment of the present invention.

FIGS. 7A and 7B illustrate a process of chemically softening the distal portion of the catheter shaft of FIG. 7.

FIG. 8 illustrates the variation in flexibility of the chemically softened distal portion of the catheter shaft of FIG. 7.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

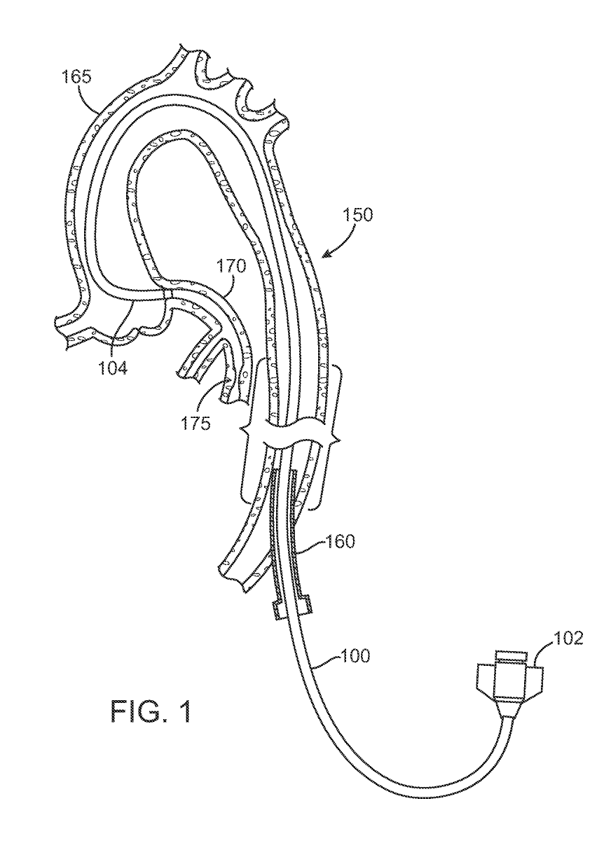

FIG. 1 illustrates guiding catheter 100 for use with a therapeutic device (not shown) positioned within a patient's vascular system 150. In a representative use of the catheter, the clinician inserts a distal end of guiding catheter 100 through introducer sheath 160 into vascular system 150, typically through a femoral artery in the groin area. Guiding catheter 100 is then advanced through aorta 165 until the distal end of the catheter is located in the ostium of a targeted branch artery 170. In the example shown, branch artery 170 is a patient's left coronary artery, and the distal end of guiding catheter 100 is positioned proximal of a stenosis 175. Once positioned, a therapeutic device, such as a balloon dilatation catheter including a dilatation balloon, may be advanced through guiding catheter 100 to provide treatment to stenosis 175. Upon completion of the interventional procedure and removal of any therapeutic device, guiding catheter 100 is withdrawn from the patient's vascular system 150.

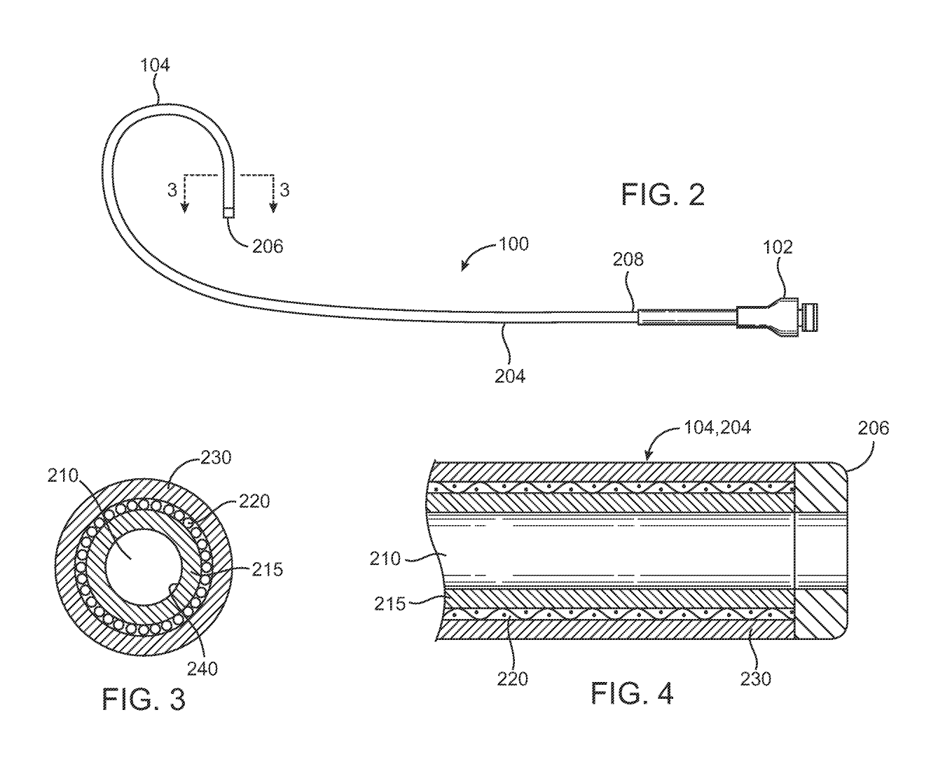

FIG. 2 illustrates a side view of an embodiment of guiding catheter 100, including an elongate shaft 204 with a distal end 206 having an optional soft tip. As shown in FIGS. 3 and 4, a bore or lumen 210 extends through shaft 204 between an open proximal end 208 and an open distal end. In an embodiment of the present invention, bore 210 has a low-friction surface 240 and is sized and shaped to receive and direct there through a variety of treatment devices, such as guidewires and/or therapeutic devices including, but not limited to balloon catheters or stent delivery systems. In another embodiment, bore surface 240 may provide a slippery interior surface for reducing frictional forces between the interior surface of guiding catheter 100 and devices that may be moved through bore 210.

A connector fitting 102 is coupled to, and provides a functional access port at the proximal end of guiding catheter 100. Fitting 102 is attached to catheter shaft 204 and has a central opening in communication with open proximal end 208 and bore 210 to allow passage of various therapeutic devices there through. Connector fitting 102 may be made of metal or of a hard polymer, e.g. medical grade polycarbonate, polyvinyl chloride, acrylic, acrylonitrile butadiene styrene (ABS), or polyamide, that possesses the requisite structural integrity, as is well known to those of ordinary skill in the art.

Catheter shaft 204 is a single lumen tubular structure that is designed to advance through a patient's vasculature to remote arterial locations without buckling or undesirable bending. In an embodiment of the present invention, catheter shaft 204 also has variable flexibility within at least distal portion 104 with its greatest flexibility proximate distal tip 206. In various other embodiments, as known to those of ordinary skill in the art, catheter shaft 204 may include a pre-formed distal curve that can provide backup support as therapeutic catheters are advanced through bore 210 of guiding catheter 100 and across stenosis 175. As shown in FIG. 2, any one of a number of pre-formed curve shapes may be incorporated into guiding catheter 100, such as Judkins-type or Amplatz-type curves, as non-limiting examples.

In the embodiment illustrated in FIGS. 2, 3 and 4, catheter shaft 204 includes an inner liner or tube 215, a reinforcing layer 220, and a continuous outer jacket or tube 230. Inner liner 215 is tubular and defines bore 210, which is sized and shaped as described above. In an embodiment of the present invention, inner liner 215 is manufactured of a high density polyethylene (HDPE) that provides good flexibility and movement of catheter 100 over a guidewire and/or movement of a therapeutic device within catheter 100. In another embodiment, inner liner 215 is manufactured of a nylon with a coating (not shown) applied to the surface of bore 210 to provide low-friction surface 240 that facilitates movement of guiding catheter 100 over a guidewire and/or movement of a therapeutic device within catheter 100. In one exemplary embodiment, the interior surface is provided with a slippery coating, such as a silicone compound or a hydrophilic polymer. Those of ordinary skill in the art may appreciate that any one of numerous low-friction, biocompatible materials such as, for example, fluoropolymers (e.g. PTFE, FEP), polyolefins (e.g. polypropylene, high-density polyethylene), or polyamides, may be used as inner liner 215 or as a coating on the surface of bore 210.

Reinforcing layer 220 enhances the torsional strength and inhibits kinking of catheter shaft 204 during advancement of guiding catheter 100 within the patient's vasculature. Reinforcing layer 220 is positioned between inner liner 215 and outer jacket 230 and is substantially coaxial with inner liner 215 and outer jacket 230. In various embodiments, reinforcing layer 220 may be formed by braiding multiple filaments or winding at least one filament over inner liner 215 or by applying a metal mesh over inner layer 215, such as a wire or mesh made from 304 stainless steel or nitinol. Braided or wound filaments may comprise high-modulus thermoplastic or thermo-set plastic materials, e.g., liquid crystal polymer (LCP), polyester, or aramid polymer e.g. poly-paraphenylene terephthalamide (Kevlar® from E.I. du Pont de Nemours and Company, Wilmington, Del., U.S.A.). Alternatively, braided or wound filaments may comprise metal wires of stainless steel, superelastic alloys, such as nitinol (TiNi), refractory metals, such as tantalum, or a work-hardenable super alloy comprising nickel, cobalt, chromium and molybdenum.

Outer jacket 230 provides support to catheter shaft 204 and coverage of reinforcing layer 220. Outer jacket 230 is coaxial with inner liner 215 and reinforcing layer 220, and is a single or unitary tube that continuously extends from proximal end 208 to distal end 206 of catheter shaft 204. In an embodiment of the present invention, outer jacket 230 is manufactured of a polyamide, such as a polyether block amide copolymer or nylon 6,6. In order to provide distal portion 104 of catheter shaft 204 with variable flexibility, at least a first distal length of outer jacket 230 is chemically softened in a softening agent for a set period of time. In another embodiment, a second distal length of outer jacket 230 may be chemically softened in the softening agent for a second period of time, which is longer than the first period of time, to achieve a greater flexibility in the second distal length versus at least a portion of the first distal length. Additional variations in flexibility of outer jacket 230 within distal portion 104 may be achieved by varying softening agent exposure time of selected distal lengths thereof, as described further below.

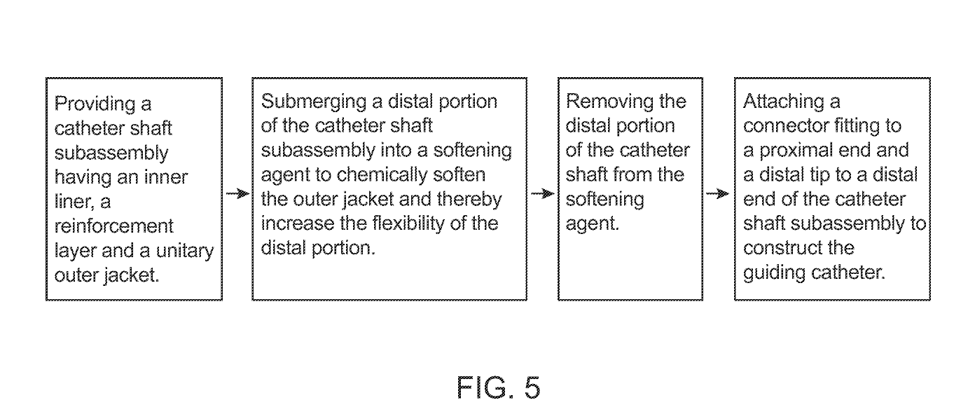

An embodiment of the present invention includes a method of manufacturing catheter shaft 204 that is selectively made more flexible by treatment with a chemical solvent. In one embodiment, as schematically illustrated in the flow chart depicted in FIG. 5, elongate reinforced layered tubing to be used for catheter shaft 204 is manufactured by first extruding an inner liner material, such as HDPE, optionally over a suitable mandrel, to form inner liner 215, which is wound continuously on a reel. Flat stainless steel wires are then selected and braided over inner liner 215 to form reinforcing layer 220, passing the long subassembly from reel to reel. An outer jacket material, such as polyethylene block amide copolymer, is then thermoplastically extruded over reinforcing layer 220 to form outer jacket 230. Outer jacket 230 may extend through the interstices of braided reinforcing layer 220 to form a bond with inner liner 215. Alternatively, an adhesive or other type of tie layer material may be incorporated to bond together inner liner 215, reinforcing layer 220, and outer jacket 230, as would be well known to those of skill in the art. The elongate reinforced layered tubing is then cut in appropriate lengths, e.g. approximately 100 cm for use in PTCA procedures performed via the femoral artery, to form a number of catheter shafts 204. If a mandrel was used during manufacturing, then it is removed from catheter shaft 204 to provide open bore 210.

At least a distal segment of distal portion 104 of each catheter shaft 204 is then chemically treated, or softened, by dipping catheter shaft distal portion 104 in a softening agent. In one embodiment, catheter shaft 204 is suspended from a rack so that a distal length/segment of approximately 20 cm of distal portion 104 is submerged in a chemical softening agent appropriate for softening the material of outer jacket 230. In an embodiment where outer jacket 230 is formed from polyethylene block amide copolymer or nylon, a dimethyl sulfoxide (DMSO) liquid has been found to be an effective and benign chemical softening agent for this purpose. Another chemical softening agent that is effective for such a catheter shaft arrangement is N,N-dimethylformamide (DMF), which may be used if the catheter shaft is properly treated after softening to neutralize any toxicity that may remain after exposure to the solvent. In another embodiment, the distal end of catheter shaft 204 may be temporarily plugged prior to the dipping process to prevent the softening agent from coming into contact with the surface of bore 210 formed by catheter shaft inner layer 215.

In an embodiment of the invention, the material(s) of inner layer 215 and outer jacket 230 may be chosen such that both are susceptible to softening with the same softening agent. In this embodiment, catheter shaft distal end 206 may remain open during the dipping process such that inner layer 215 and outer jacket 230 are both exposed to, and softened by the chemical softening agent.

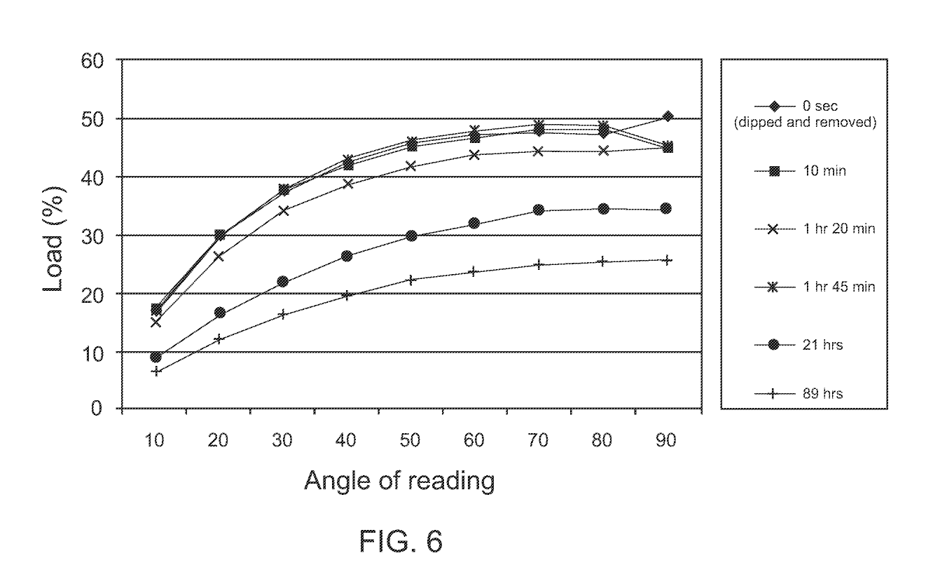

Catheter shaft distal portion 104 may be allowed to soak in the softening agent for a time period ranging from less than an hour to about 89 hours. As shown in FIG. 6, which depicts stiffness test results in a graph of load as a function of degrees of deflection for catheter shafts softened in accordance with various embodiments of the present invention, a direct correlation exists between the duration of exposure to the softening agent and a subsequent decrease in stiffness, with longer exposures correlating to increased softening of the outer jacket. During the soaking process when DMSO is used to soften an outer jacket 230 formed of a polyamide material, it is theorized that the DMSO replaces at least a portion of the hydrogen-oxygen bonds between chains of amide groups with hydrogen-oxygen bonding between amide groups and DMSO molecules. This replacement prohibits hydrogen bonding between carbonyl oxygen of one amide chain and amide hydrogen of another amide chain thus decreasing the stiffness of the material. As such, the chemical composition of the chemically softened distal portion of the catheter shaft is likely altered from that of the untreated proximal portion.

After soaking for a predetermined time period sufficient for softening outer jacket 230, catheter shaft distal portion 104 is removed from the softening agent and, optionally, wiped and/or cleaned to remove any excess softening agent. In an embodiment of the present invention, a cleaning agent, such as water, or other agent may be used that not only removes excess softening agent but also stops the softening process. A connector fitting 102 and, optionally a soft distal tip are then bonded to the proximal and distal ends 208, 206, respectively, of catheter shaft 204 to form guiding catheter 100. In a further embodiment, as shown in FIG. 2, a pre-formed curved region may be set in catheter shaft 204 by various means known to one of ordinary skill in the art.

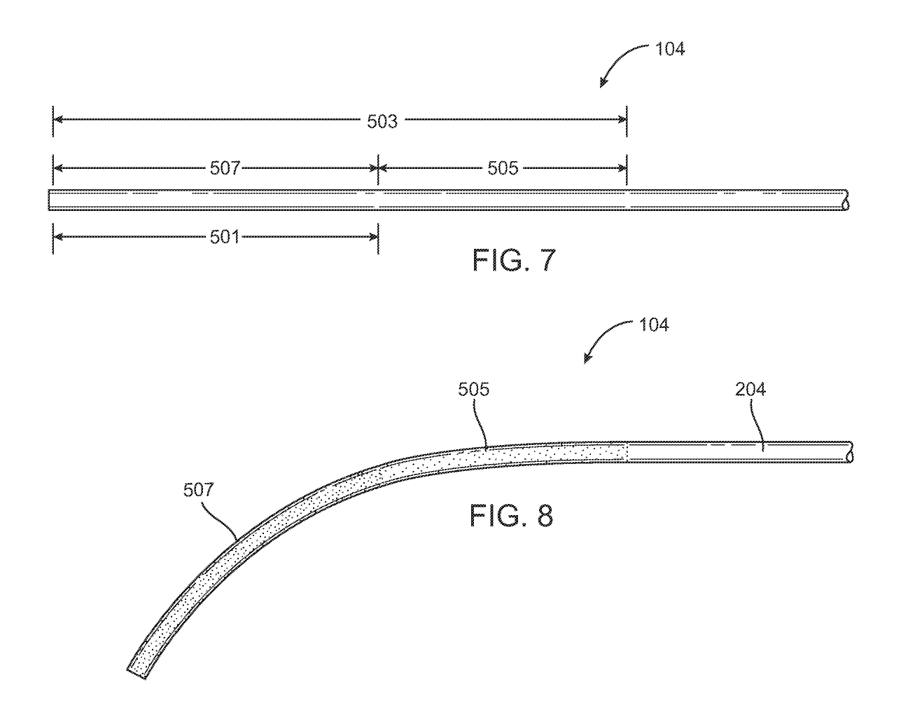

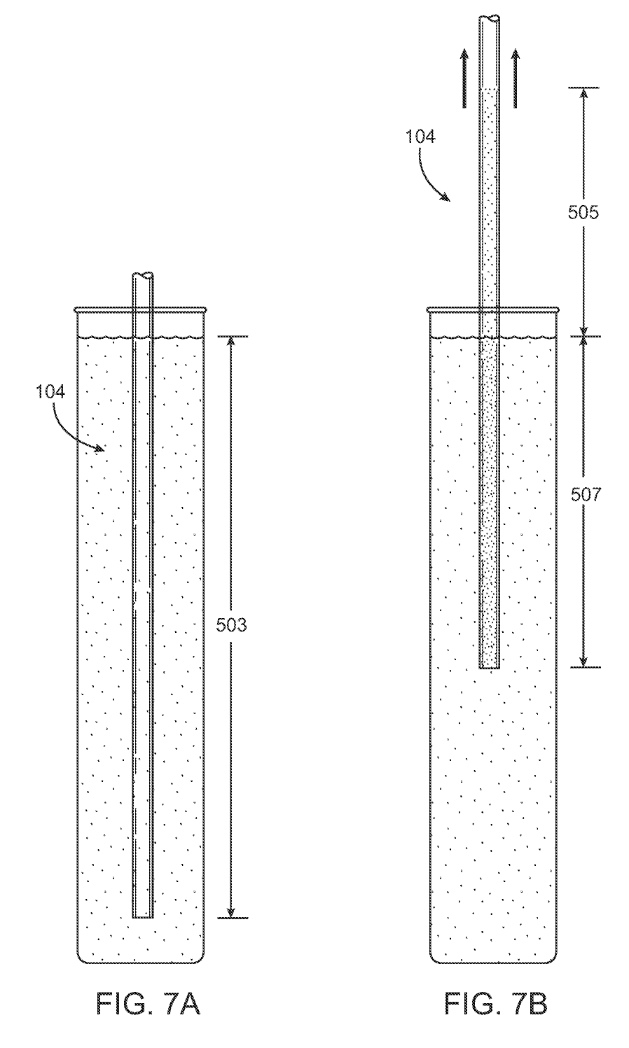

In another embodiment, as illustrated in FIGS. 7, 7A, and 7B, a first distal length 503 of distal portion 104 of catheter shaft 204, such as a first distal length up to and including 20 cm, is submerged in the softening agent for a first period of time. After soaking for the first period of time, a portion of the submerged length of catheter shaft distal portion 104 is withdrawn from the softening agent while still leaving a second distal length 501, which is a portion of first distal length 503, submerged in the softening agent for a second period of time. Upon expiration of the second period of time, second distal length 501 is removed from the softening agent, as previously discussed. A catheter shaft 204 made according to this embodiment will have three different hardnesses or stiffnesses, or described conversely, three different flexibilities. The un-submerged proximal portion of catheter shaft 204 retains its original stiffness; while a first segment 505 submerged for only the first period of time and a second segment 507 submerged for the first and second periods of time have a measurable flexibility change due to chemical softening. Because of the different time periods during which the first and second segments 505, 507 of distal portion 104 are in contact with the softening agent, each segment will have a different flexibility. As illustrated in FIG. 8, second segment 507 of catheter shaft distal portion 104 experiences the greatest change in flexibility from the untreated proximal portion of catheter shaft 204 because it was submerged in the softening agent for the longest period of time.

In further embodiments, a greater number of consecutively shorter distal lengths of distal portion 104 may be submerged for selected periods of time to create a catheter shaft distal portion 104 with more gradations in flexibility. In a still further embodiment, catheter shaft distal portion 104 may be submerged in the softening agent for a set period of time and then gradually lifted out of the softening agent at a fixed or variable rate, e.g., 1 cm/hr, or over a period of time ranging from 1 to 89 hours, until distal portion 104 is fully withdrawn from the softening agent. A distal portion 104 made according to this embodiment would have a more gradual change in flexibility along its length rather than marked or stepped changes in flexibility.

It would be understood by one of ordinary skill in the art that the variation in flexibility may also be achieved by a process in which a first distalmost length of the catheter shaft is brought into contact with a softening agent for a first period of time and then a second length of the catheter shaft, proximal to the first distalmost length, is brought into contact with the softening agent for a second period of time. In this process, the first distalmost length of the catheter shaft remains exposed to the softening agent during the first and second periods to be made more flexible than the second, more proximal, length of the catheter shaft exposed only for the second period of time. If contact in this embodiment is achieved by dipping or submerging the distal portion of the catheter in the softening agent, the depth of the dipped/submerged portion of the catheter shaft would increase over one or more periods of time until sufficient softening has occurred at which time the entire shaft would be removed from the solvent.

Those of ordinary skill in the art will recognize alternate ways to manufacture inner liner 215, reinforcing layer 220 and outer jacket 230 and that alternate materials can be utilized for each component, where the selection of a softening agent will depend on the material chosen for outer jacket 230 and/or inner liner 215. Besides the dipping processes described, selected portions of inner liner 215 and/or outer jacket 230 can be exposed to a softening agent by other processes, such as surrounding the selected portions with a sealed chamber (not shown) that can be filled with the softening agent. Such a sealed softening chamber does not expose other portions of catheter shaft 204 to the softening agent. Unlike the dipping process, a sealed chamber can be used to create segments of different flexibility wherein the segments are not necessarily arranged to provide sequentially increasing flexibility towards the distal end of catheter 100. In another embodiment, inner liner 215 may be chemically softened by aspirating a softening agent through open distal end 206 into a distal portion of bore 210, with or without exposing outer jacket 230 to the softening agent. Thus, the terms dipping or submerging are used herein to broadly describe any process wherein inner liner 215 and/or outer jacket 230 are in contact with, or exposed to a softening agent.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A guiding catheter for placement in a vessel of a patient, the catheter comprising:
an elongate hollow shaft with open proximal and distal ends, the shaft having an inner liner, a reinforcement layer overlying the inner liner, and a unitary outer jacket covering the reinforcement layer, wherein a distal portion of the outer jacket is chemically softened to be more flexible than a proximal portion of the outer jacket; and
a connector fitting mounted at the proximal end of the shaft in communication with a bore extending between the open proximal and distal shaft ends.

2. The catheter of claim 1, wherein the chemically softened distal portion includes a first softened segment with a first flexibility and a second softened segment with a second flexibility greater than the first flexibility.

3. The catheter of claim 1, wherein the unitary outer jacket is comprised of a polyamide.

4. The catheter of claim 3, wherein the polyamide is selected from the group consisting of polyethylene block amide copolymer and nylon 6,6.

5. The catheter of claim 4, wherein the outer jacket distal portion is chemically softened with dimethyl sulfoxide.

6. The catheter of claim 1, wherein the chemically softened distal portion of the outer jacket is between 2 and 20 centimeters in length.

7. The catheter of claim 1, wherein the reinforcement layer is comprised of one or more filaments that are braided or spirally wound about the inner liner.

8. The catheter of claim 7, wherein at least one filament comprises a material selected from a group consisting of a high-modulus thermoplastic, a thermo-set plastic, a liquid crystal polymer (LCP), polyester, an aramid polymer, metal, stainless steel, a superelastic alloy, nitinol (TiNi), a refractory metal, tantalum, and a work-hardenable super alloy comprising nickel, cobalt, chromium and molybdenum.

9. The catheter of claim 1, further comprising:
a soft distal tip attached to the distal end of the shaft.

10. The catheter of claim 1, wherein a distal portion of the inner liner is chemically softened to be more flexible than a proximal portion of the inner liner.

11. A method of manufacturing a guiding catheter, comprising:
providing a catheter shaft subassembly by
extruding a first material to form a tubular liner,
applying a reinforcement layer over the tubular liner, and
extruding a second material over the reinforcement layer to form an outer jacket;
contacting a distal portion of the catheter shaft subassembly with a chemical softening agent to soften at least the outer jacket and thereby increase the flexibility of the distal portion;
removing the distal portion of the catheter shaft from the softening agent; and
attaching a connector fitting to a proximal end of the catheter shaft subassembly.

12. The method of claim 11, further comprising:
plugging a distal end of the catheter shaft subassembly prior to contacting the distal portion.

13. The method of claim 11, wherein the step of removing includes removal of a first segment of the distal portion from contact with the softening agent after a first time period, such that a second segment of the distal portion remains in contact with the softening agent.

14. The method of claim 13, wherein removal of the second segment occurs upon expiration of a second time period.

15. The method of claim 11, wherein the step of removing includes slowly withdrawing the distal portion from contact with the softening agent over a period of time between 1 and 89 hours.

16. The method of claim 11, wherein the second material is a polyamide.

17. The method of claim 16, wherein the polyamide is selected from the group consisting of polyethylene block amide copolymer and nylon 6,6.

18. The method of claim 17, wherein the softening agent is dimethyl sulfoxide.

19. The method of claim 11, wherein the first and second materials are polyamides.

20. The method of claim 11, further comprising:
cleaning any residual softening agent from the distal portion of the catheter shaft after removal from contact with the softening agent.

21. The method of claim 20, wherein the cleaning step includes applying a cleaning or other agent that stops the softening process.

22. The method of claim 11, wherein the contacting step includes submerging the distal portion of the catheter shaft subassembly into the chemical softening agent.

23. The method of claim 11, wherein the contacting step includes dipping the distal portion of the catheter shaft subassembly into the chemical softening agent.

24. The method of claim 11, wherein the contacting step includes a first distalmost segment of the distal portion being in contact with the softening agent for a first time period and wherein upon expiration of the first time period, a second segment of the distal portion situated proximal to the first segment is also brought into contact with the softening agent.

25. A guiding catheter for placement in a vessel of a patient, the catheter comprising:
an elongate hollow shaft with open proximal and distal ends, the shaft having an inner liner, a reinforcement layer overlying the inner liner, and a unitary outer jacket covering the reinforcement layer, wherein a distal portion of the shaft is chemically softened to be more flexible than a proximal portion of the shaft; and
a connector fitting mounted at the proximal end of the shaft in communication with a bore extending between the open proximal and distal shaft ends.

26. The guiding catheter of claim 25, wherein a distal portion of the inner liner is chemically softened to be more flexible than a proximal portion of the inner liner.

27. The guiding catheter of claim 26, wherein a distal portion of the outer jacket is chemically softened to be more flexible than a proximal portion of the outer jacket.

28. The guiding catheter of claim 27, wherein the first and second materials are polyamides.

29. The guiding catheter of claim 28, wherein the softening agent is dimethyl sulfoxide.
